(19) United States
(12) Patent Application Publication
(10) Pub. No.: US 2008/0004575 A1
(43) Pub. Date: Jan. 3, 2008

(54) Cast/aid or cast/relief

(76) Inventor: James David McCrady, Bryan, TX (US)

Correspondence Address: James David McCrady, 511 Olive Street, Bryan, TX, 77801, US

(21) Appl. No.: 11/430,069

(22) Filed: Jul. 3, 2006

(51) Int. Cl. A61F 13/00

(52) U.S. Cl. 604/304

(57) ABSTRACT

A device which consists of a non-absorbent tape material 1 to 4 inches in width and/or an absorbent material 1 to 4 inches. Tape of the proper length and type will be determined by the attending physician. The device will eliminate major problems with hard casts that orthopedic medicine and surgery have identified: (1) lack of accessibility of the covered area for both the caregiver and patient, (2) the inability to provide medication to the underlying tissue, and (3) assistance with the psychological trauma associated with restraint.

The Cast Aid as shown and described consists of:

100 foot roll of non-absorbent 1-4 inch tape
100 foot roll of absorbent 1-4 inch tape
Tape of proper length to be inserted under the cast to relieve irritation, provide medication or reduce claustrophobia.
Stapler
Velcro or equivalent

1. I claim that the Cast Aid device is to be used in the healing arts to relieve discomfort and/or to deliver medications to the unexposed skin encompassed by a hard orthopedic cast.
